(19) United States
(12) Patent Application Publication
(10) Pub. No.: US 2015/0190372 A1
(43) Pub. Date: Jul. 9, 2015

(54) MICROCAPSULES CONTAINING RETINOIDS, METHOD OF PREPARING SAME, AND PHARMACEUTICAL COMPOSITIONS CONTAINING SAME

(71) Applicant: GALDERMA RESEARCH & DEVELOPMENT, Biot (FR)

(72) Inventor: Amel Djedour, Antibes (FR)

(21) Appl. No.: 14/404,893

(22) PCT Filed: May 30, 2013
(86) PCT No.: PCT/EP2013/061183

§ 371 Date: Dec. 1, 2014

Related U.S. Application Data
(60) Provisional application No. 61/654,723, filed on Jun. 1, 2012.

(30) Foreign Application Priority Data
Jun. 1, 2012 (FR) 1255108

(51) Int. Cl. A61K 31/402; A61K 9/50; A61K 31/192

(52) U.S. Cl. CPC A61K 31/402; A61K 31/192; A61K 9/5089; A61K 9/5057; A61K 9/5036

(57) ABSTRACT

Microcapsules are described that include a pharmaceutical active agent selected from among retinoids, an anionic hydrophilic polymer (in particular, gum arabic), and a cationic hydrophilic polymer (in particular, type-A gelatin). Also described, are methods for preparing such microcapsules, topical pharmaceutical compositions including such microcapsules, and dermatological uses thereof.

The invention relates to microcapsules comprising a pharmaceutical active agent chosen from retinoids, to processes for preparing them, to a topical pharmaceutical composition comprising these microcapsules in a physiologically acceptable medium, and to the use thereof in dermatology.

Topical treatment of moderate acne is generally the route of administration of choice and the first-line treatment, whereas, for moderate to severe acne, systemic treatment optionally combined with a topical treatment is recommended.

Several antiacne agents are available, such as antibiotics, retinoids and peroxides, each of them acting specifically on one of the physiopathological factors of acne, namely hyperkeratinization, inflammation, colonization with P. acnes and the overproduction of sebum. Among the treatment possibilities, retinoids and peroxides are the most widely used.

However, these antiacne agents have many side effects, such as skin dryness, erythema, irritation and peeling. As a result, their use poses problems of compliance with the treatment by patients. Consequently, there is a need to reduce the side effects of topically administered retinoids and peroxides.

Several formulation stratagems have been established and developed in order to reduce the restrictive side effects on patients. However, the new formulations launched on the market that have improved tolerance are few and far between.

Examples that may be mentioned are the products below containing retinoids, the tolerance of which is improved by means of a controlled release of the active principle:
adsorption of tretinoin on porous microspheres known as Microsponges®. Microsponges® are patented porous microspheres in which the active principle is adsorbed in solid form in the pores thereof. Two products containing tretinoin and using this technology exist on the U.S. market: Retin-A-Micro® 0.1% and 0.04%, approved, respectively, by the FDA in 1997 and in 2002.
introduction of a film-forming agent such as polyol prepolymer-2. This polymer makes it possible to keep the active principle dissolved or dispersed on the upper layers of the skin, limiting its penetration (Leyden, 1998). To date, three products use this technology in order to improve the tolerance of retinoids: two products with tretinoin, Avita® gel 0.025% and Avita® cream 0.025%, approved in the U.S.A. by the FDA in 1997 and 1998, and more recently Differin® lotion 0.1% with adapalene.
adsorption of adapalene onto acrylic microspheres other than Microsponges®. Clinical studies have shown that 50% of individuals who tested the new formulation reported having had side effects, as opposed to 71% in the group using the reference product (Rao et al. 2009). A new product containing adapalene based on this technology has been launched in India.

The two formulation technologies, namely adsorption and the film-forming agent, contribute toward reducing the skin irritation associated with retinoids by modulating the release kinetics of said retinoid during its application to the skin. Specifically, a delay effect is generally sought with release kinetics and thus penetration into the skin that are slower when compared with those for the same retinoid that is not absorbed or that is present in a composition free of film-forming agent.

A review of the literature also reveals other formulation technologies such as liposomes, solid lipid nanoparticles.

Schäfer-Korting et al. (1994) demonstrated that liposomes containing tretinoin at 0.01% are clinically equivalent to a commercial gel taken as reference containing 0.025% active agent. The two products show the same reduction in the number of comedones and, moreover, the liposomes are better tolerated. Patel et al. (2000) report a double-blind comparative clinical study with 30 patients over a 3-month period which demonstrates efficacy about 1.5 times superior with the liposomal formulation compared to a tretinoin gel. Furthermore, the side effects are remarkably reduced with the liposomes.

Schubert et al. (2003) describe solid lipid nanoparticles such as submicron objects between 1 and 900 nm in size, composed of lipids, allowing the incorporation of sparingly water-soluble lipophilic compounds. Preliminary irritation studies in rabbits (Draize test) have shown that tretinoin lipid nanoparticles were significantly less irritant than the commercial reference product Retin-A (Shah et al. 2007).

These novel technologies of liposome and solid lipid nanoparticle formulation make it possible to improve the tolerance of compositions containing tretinoin, but problems of tretinoin stability associated with manufacturing difficulties have limited the development of such products.

These various technologies developed with retinoids have in certain cases improved the skin tolerance, but the stability of the composition over time is not necessarily optimal. Specifically, according to these technologies, the active agent is adsorbed onto a support which places it in contact with the other ingredients of the composition. The active agent may then be unstable in the composition, which may lead to instability of the composition.

Moreover, slower release kinetics may have an impact on the efficacy of the retinoid. Specifically, the amount of retinoid available to be absorbed into the skin, and thus the concentration present in the skin tissues, may be below the minimum effective concentration for obtaining the therapeutic effect.

It is therefore necessary to develop novel pharmaceutical compositions containing active agents that are well tolerated, which have release kinetics that ensure an effective therapeutic concentration and which have prolonged physical and chemical stability over time.

According to the invention, the term “physical stability” refers to a composition whose physical properties such as the organoleptic properties, pH and viscosity are stable over time and under various temperature conditions: 4° C., room temperature, 40° C.

According to the invention, the term “chemical stability” refers to a composition in which the active principle is chemically stable over time, irrespective of the temperature condition: 4° C., room temperature, 40° C.

The Applicant has thus discovered a novel topical pharmaceutical composition containing an active agent, such as retinoids, held in microcapsules, which allows an improvement in tolerance, and in particular a reduction in irritation, while at the same time showing good physical and chemical stability of the retinoids and of the composition as a whole.

Specifically, the Applicant has shown, surprisingly, that, by means of this particular encapsulation technique, these dissolved or dispersed active agents are protected by the microcapsules from the other ingredients of the composition. Specifically, the use of the microcapsules according to the present invention in pharmaceutical compositions for topical use makes it possible to improve the chemical and physical stability of the final compositions, when the active agent degrades in the presence of other excipients present in the composition.

The pharmaceutical compositions according to the invention containing these microcapsules also allow a controlled release of the active agent in two phases:
A first release phase with a delay effect taking place immediately after the application, making it possible to reduce the concentration of the retinoid responsible for the irritation phenomena, generally due to an excessive amount of retinoid released immediately after the application, The first phase also has slower release kinetics than the second release phase.
A second release phase with kinetics identical to those of the same, non-encapsulated retinoid. The second phase has the advantage of not reducing the amount of retinoid available to be absorbed into the skin and thus of reducing the effective therapeutic concentration of the retinoid.

The invention will be described in greater detail in the description and the examples which follow, and also in the appended figures in which:

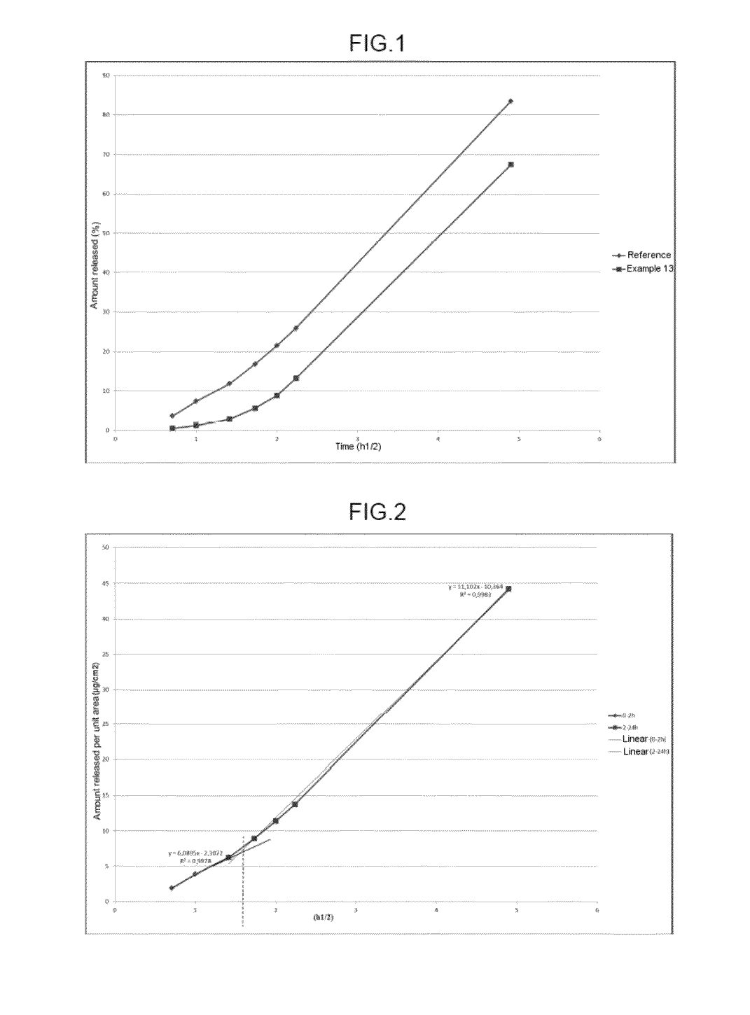

FIG. 1 shows the amount released in percentage of a preferred retinoid in the present invention (“compound A”) as a function of the square root of the time for a reference gel and for a composition according to the invention.

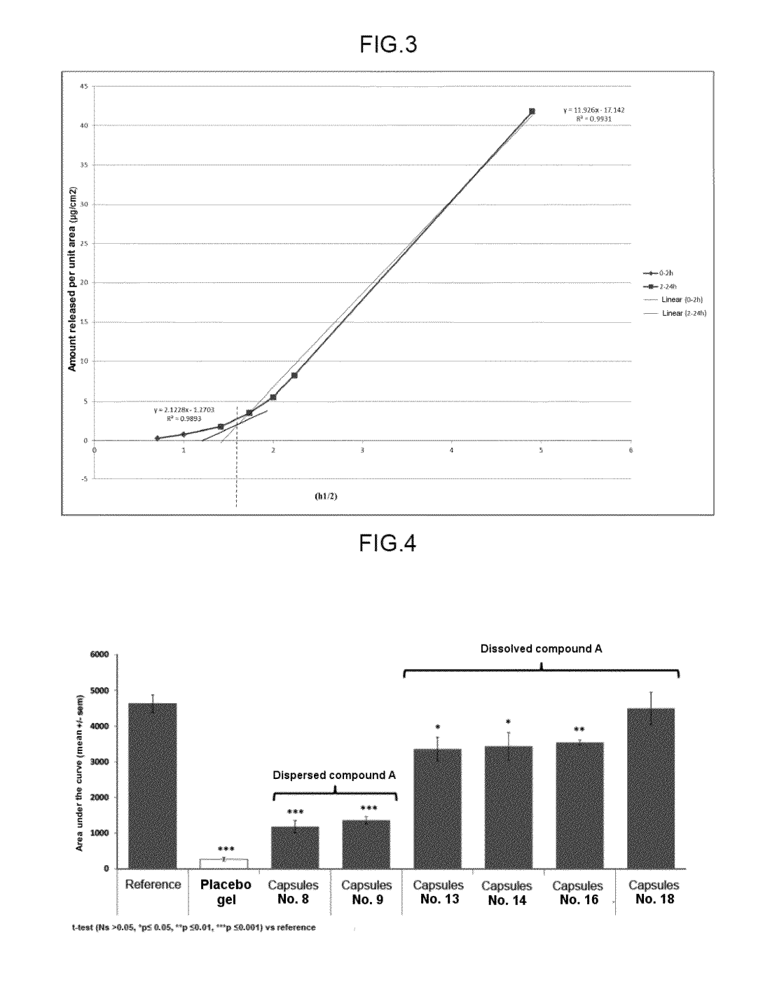

FIGS. 2 and 3 show, respectively, the amount of compound A expressed in μg/cm² as a function of the square root of the time for a composition according to the invention.

FIG. 4 shows the results of a tolerance study performed on a reference gel, a placebo gel and compositions according to the invention.

One subject of the present invention is microcapsules obtained by complex coacervation, which comprise a pharmaceutical active agent, for example a retinoid.

Complex coacervation is an encapsulation technique. It allows the production of microcapsules or coacervates by formation of a polymer layer around a lipophilic core which may be oil droplets or solid particles.

This technology applied to active agents and more particularly to retinoids allows a controlled release thereof in two phases by diffusion through the polymer layer in order to improve the tolerance. The term “controlled release” means a release of a regular dose of the active agent over time. The term “release phase” means release kinetics with a defined release constant.

Depending on its solubility parameters, the active agent in the microcapsules may be encapsulated either directly in the solid state in the form of solid particles, or dispersed in a fatty phase, or dissolved in a fatty phase.

In the case where the active agent is dispersed, the encapsulation may be performed either directly on the solid particles or on these same solid particles dispersed in a non-solvent liquid phase. The term “liquid phase” means a phase that is not solid at room temperature. This liquid phase is generally water-immiscible.

According to the present invention, the microcapsules are obtained by means of a polymer layer formed around oil droplets containing the active agent or solid active agent particles. This polymer layer consists of two hydrophilic biopolymers of opposite charge.

Complex coacervation corresponds to the simultaneous desolvatation of two oppositely charged polymers of water-soluble polyelectrolyte type, brought about following a modification of the pH of the reaction medium and the induced electrostatic attraction of the two polymers.

These complexes aggregate and form droplets known as coacervates.

Once the coacervate has formed and become deposited around the oil droplets containing the active agent, a crosslinking agent is added so as to solidify this coacervate and thus to form microcapsules.

The term “microcapsules” means objects of micrometric size consisting of a membrane or envelope coating a central part which may be liquid or solid at room temperature. The microcapsules function as reservoir systems, and thus the retinoid(s) encapsulated in the microcapsules are released by diffusion through the membrane or envelope surrounding this core or by rupture of the membrane or envelope due to shear during the application to the skin.

The microcapsules according to the invention are small, ideally less than 120 μm, preferably less than 60 μm and ideally about 20 μm.

According to a first variant of the invention, the microcapsules comprise:
a pharmaceutical active agent chosen from retinoids,
a cationic hydrophilic polymer chosen from gelatins of type A, and
an anionic hydrophilic polymer.

According to a second variant of the invention, the microcapsules comprise:
3″-tert-butyl-4′-(2-hydroxyethoxy)-4″-pyrrolidin-1-yl-[1,1′;3′,1″]-terphenyl-4-carboxylic acid as active agent, in solid form or in dispersed form in a lipophilic phase,
a cationic hydrophilic polymer, and
an anionic hydrophilic polymer,
   and are characterized in that the area under the curve, determined by applying to the ears of mice, once a day for 4 consecutive weeks, 3 mg of a composition containing said microcapsules, such that the content of 3″-tert-butyl-4′-(2-hydroxyethoxy)-4″-pyrrolidin-1-yl-[1,1′;3′,1″]-terphenyl-4-carboxylic acid is 0.01% by weight relative to the total weight of the composition, and by measuring the thickness of the mouse ear from day 2 and then daily up to day 26, and plotting the corresponding graph representing the change in thickness of the ear over time and calculating the area under this curve, is less than 2000 μm per day.

Preferably, in this second variant, the area under the curve is between 1000 and 2000 μm per day.

According to a third variant of the invention, the microcapsules comprise:
3″-tert-butyl-4′-(2-hydroxyethoxy)-4″-pyrrolidin-1-yl-[1,1′;3′,1″]-terphenyl-4-carboxylic acid as active agent, in dissolved form in a lipophilic phase,
a cationic hydrophilic polymer,
an anionic hydrophilic polymer,
   and are characterized in that the area under the curve, determined by applying to the ears of mice, once a day for 4 consecutive weeks, 3 mg of a composition containing said microcapsules, such that the content of 3″-tert-butyl-4′-(2-hydroxyethoxy)-4″-pyrrolidin-1-yl-[1,1′;3′,1″]-terphenyl-4-carboxylic acid is 0.01% by weight relative to the total weight of the composition, and by measuring the thickness of the mouse ear from day 2 and then daily up to day 26, and plotting the corresponding graph representing the change in thickness of the ear over time and calculating the area under this curve, is less than 4000 μm per day.

Preferably, in this third variant, the area under the curve is between 3000 and 4000 μm per day.

In the second and third embodiment variants above, for the determination of the area under the curve characterizing the microcapsules according to the invention, it is possible to proceed by incorporating the microcapsules, for example, in a composition containing the following ingredients:

| | Composition |
|---|---|
| | (weight percentage, |
| | relative to the |
| Ingredients | total weight) |
| Sodium docusate | 0.05 |
| Sodium edetate | 0.10 |
| Methyl paraben | 0.20 |
| Glycerol | 4.00 g |
| 1,2-Propanediol | 4.00 |
| Poloxamer 124 | 0.20 |
| Acrylamide/AMPS copolymer | 4.00 |
| (as a 40% dispersion in | |
| isohexadecane) | |
| Purified water | qs 100 |

The retinoids that may be used in the context of the invention especially comprise all-trans-retinoic acid or tretinoin, 13-cis-retinoic acid or isotretinoin, acitretin, arotinoic acid, retinol, adapalene, tazarotene, retinaldehyde, etretinate and the compounds protected in patent application WO 2006/066 978 such as 3″-tert-butyl-4′-(2-hydroxyethoxy)-4″-pyrrolidin-1-yl-[1,1′;3′,1″]-terphenyl-4-carboxylic acid, the compounds of patent application FR 05/12367 including 2-hydroxy-4-[3-hydroxy-3-(5,6,7,8-tetrahydro-5,5,8,8-tetramethyl-2-naphthyl)-1-propynyl]benzoic acid or an enantiomer thereof, the compounds of patent application WO 05/56516 including 4′-(4-isopropylamino-butoxy)-3′-(5,5,8,8-tetramethyl-5,6,7,8-tetrahydronaphthalen-2-yl)-biphenyl-4-carboxylic acid, the compounds of patent application PCT/EP04/014809 including 4-{3-hydroxy-3-[4-(2-ethoxyethoxy)-5,5,8,8-tetramethyl-5,6,7,8-tetrahydronaphthalen-2-yl]-prop-1-ynyl}benzoic acid, and the compounds of patent application FR 2 861 069 including 4-[2-(3-tert-butyl-4-diethylaminophenyl)-2-hydroxyiminoethoxy]-2-hydroxybenzoic acid.

3″-tert-Butyl-4′-(2-hydroxyethoxy)-4″-pyrrolidin-1-yl-[1,1′;3′,1″]-terphenyl-4-carboxylic acid as protected in patent application WO 2006/066 978, referred to as “compound A” in the rest of the present patent application and adapalene are particularly preferred.

The term “cationic hydrophilic polymer” (or “cationic macromolecule”) means a polymer that has been made cationic by reducing the pH below its isoelectric point.

The positively charged macromolecule is advantageously chosen from cationic biopolymers such as polypeptides, proteins or polysaccharides.

As examples of biopolymers of cationic protein type, mention may be made in a nonlimiting manner of gelatin of type A whose isoelectric point is between pH 7-9, obtained by partial acid hydrolysis, such as the product sold by the company Weishardt International under the name Gelatine 280 Bloom 20 Mesh.

As examples of biopolymers of cationic polysaccharides type, mention may be made of chitin derivatives such as high molecular weight chitosans, which are cationic at pH 6.5, with a high degree of deacetylation, such as the products sold by the company Chitinor under the name Chitopharm®. The cationic polymer according to the present invention is preferentially gelatin of type A.

The anionic hydrophilic polymer is advantageously chosen from anionic biopolymers such as polypeptides, proteins or polysaccharides.

Examples of biopolymers of anionic protein type that may be mentioned include gelatin of type B obtained by partial alkaline hydrolysis and whose isoelectric point is between pH 4.7-5.4.

As examples of biopolymers of anionic polysaccharide type, nonlimiting examples that may be mentioned include gum arabic or acacia, the gellan gum sold under the name Kelcogel by the company Kelco, alginates such as the sodium alginate sold under the name Satialgine® by the company Cargill; carrageenans such as those sold by the company IMCD under the names Gelcarin® and Viscarin® (for example: Gelcarin GP812N®, Gelcarin GP379NF®, Viscarin GP209Nr).

The anionic polymer according to the present invention is preferentially gum arabic.

The key parameter for formation of the polymer layer is the pH variation. Specifically, a decrease in pH below the isoelectric point of the hydrophilic polymer makes this polymer cationic, which, as a result, interacts with the anionic hydrophilic polymer, at this pH value. A pH regulator is thus introduced into the preparation.

In the present invention, the term “pH regulator” means an acid for reducing the pH of the preparation to the isoelectric point of the two polymers, such that these polymers are of opposite charge and can form the coacervation complexes.

Preferentially, the coacervation pH for this embodiment is from 4.9 to 5.0.

As a nonlimiting example, this acid may be acetic acid.

The pH corrector is then removed at the end of preparation of the microcapsules during the successive washing.

The microcapsules according to the invention advantageously comprise at least one crosslinking agent, which allows the formation of covalent bonds between said ionic hydrophilic polymer and said cationic hydrophilic polymer.

As crosslinking agent, mention may be made in a nonlimiting manner of transglutaminase, tannic acid, an aldehyde or a derivative thereof such as formaldehyde or glutaraldehyde, or mixtures thereof.

Preferentially, the crosslinking agent according to the present invention is glutaraldehyde.

This crosslinking agent allows the formation of covalent bonds of amide type via the chemical reaction of the amine groups of the protein with the carboxylic groups of the polysaccharide. At the end of reaction, the residual glutaraldehyde is removed by successive washing of the microcapsules.

According to a first particularly preferred embodiment of the invention, the microcapsules according to the invention comprise:
compound A,
gelatin of type A, and
gum arabic.

Compound A in dispersed form in the microcapsules is preferably present in a concentration ranging from 0.001% to 1% and more preferentially ranging from 0.1% to 0.7% by weight relative to the total weight of the microcapsules.

Compound A in dissolved form in the microcapsules is preferably present in a concentration ranging from 0.001% to 0.5% and more preferentially ranging from 0.1% to 0.3% by weight relative to the total weight of the microcapsules.

Among the solvents for compound A, mention may be made especially of triglycerides, for instance the capric/caprylic acid triglycerides mixture sold under the name Miglyol® 812N, fatty acid esters, for instance the diisopropyl adipate sold under the name Crodamol® DA by the company Croda, polyethoxylated fatty acids, for instance the oleoyl macrogol-6 and glycerides sold under the name Labrafil® M1944CS by the company Gattefossé, fatty alcohols, for instance the octyldodecanol sold under the name Eutanol® G, fatty alkyl esters, glycols and derivatives, and glycol ethers, for instance the PPG-15 stearyl ether sold under the name Arlamol® PSE15 by the company Croda.

According to a second also preferred embodiment of the invention, the microcapsules according to the invention comprise:
adapalene,
gelatin of type A, and
gum arabic.

Adapalene in dispersed form in the microcapsules is preferably present in a concentration ranging from 0.01% to 10% and more preferentially ranging from 3% to 7% by weight relative to the total weight of the microcapsules.

The microcapsules according to the invention may also contain a lipophilic phase (or fatty phase or oily phase) chosen from:
solvents that are suitable for the retinoid active agent, when it is encapsulated in dissolved form,
fatty phases that are non-solvents for the active agent, when the active agent is encapsulated in dispersed form.

This lipophilic phase may comprise, for example, plant oils, mineral oils, animal oils, synthetic oils or silicone oils, and mixtures thereof.

As examples of mineral oils, mention may be made, for example, of liquid paraffins of various viscosities, such as Primol 352® and Marcol 152® sold by the company Univar.

As plant oils, mention may be made of sweet almond oil (Prunus amygdalus dulcis) sold by Sictia, palm oil, soybean oil, sesame oil, sunflower oil and olive oil.

As animal oils, mention may be made of lanolin, squalene, fish oil with, as a derivative, the perhydrosqualene sold under the name Sophiderm® by the company Sophim.

As synthetic oils, mention may be made of an ester such as cetearyl isononanoate, for instance the product sold under the name Cetiol SN PH® by the company Chitinor France, diisopropyl adipate, for instance the product sold under the name Crodamol DA® by the company Croda, isopropyl palmitate, for instance the product sold under the name Crodamol IPP® by the company Croda, and caprylic/capric triglyceride, such as Miglyol 812® sold by the company Univar.

As silicone oils, mention may be made of a dimethicone, for instance the product sold under the name Q7-9120 Silicone Fluid® with a viscosity of 20 cSt to 12 500 cSt, by the company Dow Corning, or a cyclomethicone, for instance the product sold under the name ST-Cyclomethicone 5NF®, also by the company Dow Corning.

As examples of lipophilic phases, mention may also be made of propylene glycol monocaprylate (Capryol® 90) sold by Gattefossé, propylene glycol laurate (Lauroglycol® FCC) sold by Gattefossé, diisopropyl adipate (Crodamol® DA) sold by Croda, PPG-15 stearyl ether (Arlamol® PS15E) sold by Croda, and apricot kernel oil PEG-6 ester or oleoyl macrogol-6 glyceride (Labrafil® M1944CS).

When the complex coacervation is performed around oil droplets in which the active principle is dispersed or dissolved, the polymer/oil weight ratio, i.e. the total weight amount of cationic hydrophilic polymer added to that of anionic hydrophilic polymer over the total amount of lipophilic phase, is advantageously between 0.2 and 0.8 and preferentially between 0.3 and 0.5.

The microcapsules may also contain additives for improving their stability. Mention may be made of additives such as suspension agents, gelling agents or preserving agents.

Nonlimiting examples of the intended suspension agents and gelling agents include Acrylates/C10-30 alkyl acrylate crosspolymer sold under the name Pemulen TR1 or Pemulen TR2 by the company Lubrizol, the carbomers sold under the name Ultrez 20®, Ultrez 10®, Carbopol 1382® or Carbopol ETD2020NF®, Carbopol® 981 or Carbopol® 980 by the company Lubrizol, polysaccharides, nonlimiting examples being xanthan gum such as Xantural 180® sold by the company Kelco or Satiaxane® UCX 911 sold by Cargill, polyvinyl alcohol such as Polyvinyl alcohol 40-88 sold by Merck, gellan gum sold under the name Kelcogel by the company Kelco, guar gum, cellulose and derivatives thereof such as the microcrystalline cellulose and sodium carboxymethylcellulose sold under the name Avicel® CL-611 by the company FMC Biopolymer, hydroxypropylmethylcellulose, in particular the product sold under the name Methocel® E4M premium by the company Dow Chemical, or hydroxyethylcellulose, in particular the product sold under the name Natrosol HHX 250® by the company Aqualon, the family of aluminum magnesium silicates such as Veegum® K sold by the company Vanderbilt, the family of acrylic polymers coupled to hydrophobic chains such as PEG-150/decyl/SMDI copolymer sold under the name Aculyn® 44 (polycondensate comprising at least, as elements, a polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)), the family of modified starches such as the modified potato starch sold under the name Structure Solanace, or mixtures thereof, and gelling agents of the family of polyacrylamides, such as the mixture Sodium acryloyldimethyltaurate copolymer/isohexadecane/polysorbate 80 sold under the name Sepineo P600® (or Simulgel 600 PHA®) by the company SEPPIC, the mixture polyacrylamide/isoparaffin C13-14/laureth-7, for instance the product sold under the name Sepigel® 305 by the company SEPPIC, the family of carrageenans, in particular divided into four major families: κ, λ, β, ω such as the Viscarin® products and the Gelcarin® products sold by the company IMCD.

Nonlimiting examples of the intended preserving agents include methyl paraben such as Nipagin® M sold by Clariant, propyl paraben, benzalkonium chloride, phenoxyethanol sold under the name Phenoxetol® by Clariant, benzyl alcohol sold under the name benzyl alcohol by Merck, sodium benzoate sold under the name Probenz® SP by Unipex, potassium sorbate sold under the name potassium sorbate by VWR, benzoic acid sold under the name benzoic acid by VWR, 2-bromo-2-nitropropane-1,3-diol sold under the name Bronopol® by Jan Dekker International, chlorhexidine sold under the name Chlorexidine digluconate 20% solution by Arnaud Pharmacie, chlorocresol and derivatives thereof, ethyl alcohol and diazolidinylurea. These preserving agents may be used alone or in combination in order to efficiently protect the formulae against any bacterial contamination.

The microcapsules of the present invention are advantageously used for preparing the pharmaceutical compositions for topical use.

A subject of the present invention is thus also a topical pharmaceutical composition containing the microcapsules described above, obtained by complex coacervation comprising a pharmaceutical active agent, such as retinoids.

Preferentially, the pharmaceutical active agent included in the compositions according to the invention will be a retinoid.

The compositions according to the present invention may be in any galenical form normally used for topical application, especially in the form of aqueous, aqueous-alcoholic or oily dispersions, suspensions, aqueous, anhydrous or lipophilic gels, emulsions (lotions, creams or pomades) of liquid, semi-solid or solid consistency, obtained by dispersing a fatty phase in an aqueous phase (oil-in-water emulsions) or conversely (water-in-oil emulsions) in the presence or absence of an emulsifier, or alternatively microemulsions.

Preferably, the compositions according to the invention are in the form of emulsions (lotions, creams or emulsifier-free creams), suspensions or gels, and more preferentially in the form of gels and emulsions.

In the compositions according to the invention, when the retinoid is adapalene, it is advantageously present in a concentration ranging from 0.001% to 10% by weight and preferentially from 0.01% to 5% by weight relative to the total weight of the composition.

When the retinoid is compound A, it is advantageously present in a concentration ranging from 0.00001% to 1% by weight and preferentially from 0.0001% to 0.1% by weight relative to the total weight of the composition.

The composition according to the invention may also comprise one or more gelling agents. As nonlimiting examples of gelling agents that may be included in the compositions according to the invention, mention may be made of Acrylates/C10-30 alkyl acrylate crosspolymer sold under the name Pemulen® TR1 or Pemulen® TR2 by the company Lubrizol, the carbomers sold under the name Ultrez 20®, Ultrez 10®, Carbopol 1382® or Carbopol ETD2020NF®, Carbopol® 981 or Carbopol® 980 by the company Lubrizol, polysaccharides, nonlimiting examples being xanthan gum such as Xantural 180® sold by the company Kelco or Satiaxane® UCX 911 sold by Cargill, polyvinyl alcohol such as Polyvinyl alcohol 40-88 sold by Merck, gellan gum sold under the name Kelcogel by the company Kelco, guar gum, cellulose and derivatives thereof such as microcrystalline cellulose and sodium carboxymethyl-cellulose sold under the name Avicel® CL-611 by the company FMC Biopolymer, hydroxypropylmethylcellulose, in particular the product sold under the name Methocel® E4M premium by the company Dow Chemical, or hydroxyethylcellulose, in particular the product sold under the name Natrosol HHX 250® by the company Aqualon, the family of aluminum magnesium silicates such as Veegum® K sold by the company Vanderbilt, the family of acrylic polymers coupled to hydrophobic chains such as PEG-150/decyl/SMDI copolymer sold under the name Aculyn® 44 (polycondensate comprising at least, as elements, a polyethylene glycol containing 150 or 180 mol of ethylene oxide, of decyl alcohol and of methylenebis(4-cyclohexyl isocyanate) (SMDI), at 35% by weight in a mixture of propylene glycol (39%) and water (26%)), the family of modified starches such as the modified potato starch sold under the name Structure Solanace, or mixtures thereof, and gelling agents of the family of polyacrylamides, such as the mixture Sodium acryloyldimethyltaurate copolymer/isohexadecane/polysorbate 80 sold under the name Sepineo P600® (or Simulgel 600 PHA®) by the company SEPPIC, the mixture polyacrylamide/isoparaffin C13-14/laureth-7, for instance the product sold under the name Sepigel® 305 by the company SEPPIC, the family of carrageenans, in particular divided into four major families: κ, λ, β, ω such as the Viscarin® products and the Gelcarin® products sold by the company IMCD.

The composition according to the invention may also comprise a fatty phase, which may consist, as nonlimiting examples, of:
one or more mineral oils, for instance liquid paraffins of different viscosities, for instance Marcol® 152, Marcol® 52 or Primo® 352 sold by Univar,
one or more plant oils, among which mention may be made of sweet almond oil, palm oil, soybean oil, sesame oil, sunflower oil, hydrogenated castor oil or coconut oil,
one or more synthetic oils, among which mention may be made of apricot kernel oil PEG-6 ester (Labrafil® M1944CS), propylene glycol laurate (Lauroglycol® FCC), propylene glycol monocaprylate (Capryol® 90) sold by Gattefossé, esters such as cetearyl isononanoate, for instance the product sold under the name Kollicream® CL by the company BASF France, and isopropyl palmitate, for instance the product sold under the name Crodamol® IPP by the company Croda,
one or more animal oils, among which mention may be made of lanolin, squalene, fish oil, mink oil, with, as a derivative, the squalane sold under the name Cosbiol® by the company Laserson,
one or more silicone oils for improving the properties of the formula on application, such as cyclomethicone (St-Cyclomethicone® 5NF) or dimethicone (Q7 9120 silicon fluid having a viscosity of 20 cSt to 12 500 cSt from Dow Corning),
one or more fatty-phase thickeners of fatty alcohol type, such as cetyl alcohol (Crodacol® C70 supplied by Croda/Lanette® 16 sold by BASF, but also Kolliwax® CA sold by BASF), cetearyl alcohol (Crodacol® 1618 sold by Croda, Tego Alkanol® 1618 sold by Evonik, but also Kolliwax® CSA sold by BASF), stearyl alcohol (Crodacol® S95 sold by Croda, Kolliwax® SA sold by BASF, but also Tego Alkanol® 18 sold by Evonik), but also behenyl alcohol (Lanette® 22 sold by BASF, Nacol® 22-98 sold by Sasol, but also Behenyl Alcohol® 65 80 sold by Nikko Chems), or of carnauba wax type sold by Baerlocher, but also the beeswax sold under the name Cerabeil Blanchie DAB® sold by Univar, and glyceryl tribehenate such as Compritol® 888 sold by Gattefossé. In this case, a person skilled in the art will adjust the heating temperature of the preparation according to the presence or absence of these solids.

Other oils or fatty substances may be added to the fatty phase of the composition in a varied manner by a person skilled in the art in order to prepare a composition having the desired properties, for example in terms of consistency or texture.

Thus, when the composition according to the invention is in emulsion form, the fatty phase may be present in a content ranging from 1% to 95% by weight relative to the total weight of the composition, preferably from 5% to 85% and more preferentially from 15% to 50% by weight relative to the total weight of the composition.

The composition according to the invention may also contain additives or combinations of additives, such as:
surfactants;
pro-penetrants;
stabilizers;
humectants;
humidity regulators;
pH regulators;
osmotic pressure modifiers;
chelating agents;
preserving agents;
UV-A and UV-B screening agents;
and antioxidants.

Needless to say, a person skilled in the art will take care to select the optional compound(s) to be added to these compositions such that the advantageous properties intrinsically associated with the present invention are not, or are not substantially, adversely affected by the envisaged addition.

These additives may be present in the composition in contents ranging from 0 to 40% by weight relative to the total weight of the composition.

A subject of the present invention is also the process for preparing the microcapsules described above.

The process for preparing the microcapsules according to the invention comprises the following steps:
dissolution of the two oppositely charged hydrophilic polymers;
addition of the retinoid and mixing of the two phases;
addition of the pH regulator to the coacervation pH;
addition of a crosslinking agent;
drying of the microcapsules;
removal of the crosslinking agent by washing with a saline solution;
successive washing of the preparation with water and drying.

When the retinoid is in the solid state, and encapsulated in the form of solid particles, it may be incorporated directly into the hydrophilic polymer solution, before the addition of the second polymer. When the retinoid is dispersed or dissolved in a lipophilic phase, it is incorporated into the mixtures of the two oppositely charged hydrophilic polymers.

A subject of the present invention is also the use of a composition according to the invention for treating one or more of the following pathologies:

1) dermatological conditions associated with a keratinization disorder relating to cell differentiation and proliferation, in particular for treating common acne, comedonal acne, polymorphic acne, acne rosacea, nodulocystic acne, acne conglobata, senile acne, secondary acne such as solar acne, acne medicamentosa or occupational acne;

2) keratinization disorders, in particular ichthyosis, ichthyosiform conditions, lamellar ichthyosis, Darier's disease, palmoplantar keratoderma, leukoplakia, pityriasis rubra pilaris and leukoplakiform conditions, cutaneous or mucosal (buccal) lichen;

3) dermatological conditions with an inflammatory immuno-allergic component, with or without a cell proliferation disorder, and in particular all forms of psoriasis, whether cutaneous, mucosal or ungual, and even psoriatic arthritis, or else atopic dermatitis and the various forms of eczema;

4) skin disorders caused by exposure to UV radiation, and also for repairing or combating skin aging, whether it is photo-induced or chronological, or for reducing actinic keratoses and pigmentations, or any pathological conditions associated with chronological or actinic aging, such as xerosis, pigmentations and wrinkles;

5) conditions associated with benign dermal or epidermal proliferations, whether or not they are of viral origin, such as common warts, flat warts, molluscum contagiosum and epidermodysplasia verruciformis, or oral or florid papillomatoses;

6) dermatological disorders such as immune dermatoses, for instance lupus erythematosus, bullous immune diseases and collagen diseases, such as scleroderma;

7) stigmata of epidermal and/or dermal atrophy induced by local or systemic corticosteroids, or any other form of cutaneous atrophy;

8) cicatrization disorders, or for preventing or repairing stretch marks, or else for promoting cicatrization;

9) skin complaints of fungal origin, such as tinea pedis and tinea versicolor;

10) pigmentation disorders, such as hyperpigmentation, melasma, hypopigmentation or vitiligo;

11) cutaneous or mucosal cancerous or precancerous conditions, such as actinic keratoses, Bowen's disease, in-situ carcinomas, keratoacanthomas and skin cancers such as basal cell carcinoma (BCC), squamous cell carcinoma (SCC) and cutaneous lymphomas such as T lymphoma.

The pharmaceutical composition is preferentially intended for treating: acne, ichthyosis, ichthyosiform conditions, palmoplantar keratosis, psoriasis.

A subject of the present invention is thus also a composition as described above, for its use for treating the pathologies described above.

### EXAMPLES

### Processes for Obtaining the Microcapsules:

The following process examples are given in a nonlimiting manner, for the preparation of microcapsules according to the invention.

The stirring speeds and times used are adjusted so as to allow the production of microcapsules of the desired size.

### Process Example 1

### Production of Microcapsules With Solid Retinoid Encapsulated

Heat the dilution water to 40° C. in a reactor.
Prepare the gum arabic solution in a formulation beaker of suitable size. Disperse the retinoid in this phase and heat to 40° C.
Prepare the aqueous solution of gelatin of type A in a second beaker. Heat to 40° C. The aqueous phase is heated so as to promote the dissolution of the two hydrophilic polymers.
With stirring, gently pour the solution of gelatin of type A into the aqueous solution of gum arabic containing the dispersed retinoid. Keep stirring until the mixture is fully homogeneous.
Next, perform dilution in the reactor, with the dilution water at 40° C.
With stirring, add acetic acid to the preparation in an amount sufficient to descend to the coacervation pH (pH=4.9 in the case of the present invention).
Next, decrease the temperature to 10° C. to obtain gelation of the coating.
Solidify the coacervates by adding the crosslinking agent (e.g. glutaraldehyde).
Dry at 50° C.
Recover and wash the capsules in a specific saline solution so as to remove the residual glutaraldehyde.
Wash twice more with water so as to remove the residual salts.
Next, add the preserving agent to the preparation.

Dry the coacervates under a gentle vacuum to obtain a manipulable capsule paste.

The following characterizations were performed:
Karl Fischer measurement of the residual water content
the solids content is determined by gravimetry after total evaporation of the water
measurement of the particle size using a laser particle size analyzer of Malvern type

### Process Example 2

### Production of Microcapsules With Retinoid Dispersed or Dissolved in a Lipophilic Phase

Heat the dilution water to 40° C. in a reactor.
Prepare the aqueous solution of polymers (gum arabic and gelatin of type A) in a formulation beaker of suitable size. Heat the mixture to 40° C. The aqueous phase is heated so as to promote the dissolution of the two hydrophilic polymers.
In a second beaker, disperse or dissolve the retinoid in the lipophilic phase. Heat to 40° C.
With stirring, gently pour the lipophilic phase containing the retinoid into the aqueous solution of polymer. Keep stirring until the mixture is fully homogeneous (emulsification).
Next, perform dilution of the emulsion in the reactor, with the dilution water at 40° C.
With stirring, add acetic acid to the emulsion in an amount sufficient to descend to the coacervation pH (pH=4.9 in the case of the present invention).
Next, decrease the temperature to 10° C. to obtain gelation of the coating.
Solidify the coacervates by adding the crosslinking agent (e.g. glutaraldehyde).
Dry at 50° C.
Recover and wash the microcapsules in a specific saline solution so as to remove the residual glutaraldehyde.
Wash twice more with water so as to remove the residual salts.
Next, add the preserving agent to the preparation.
Dry the coacervates under a gentle vacuum to obtain a manipulable microcapsule paste.

The following characterizations were performed:
Karl Fischer measurement of the residual water content
the solids content is determined by gravimetry after total evaporation of the water
the amount of oil corresponds to the sum of the compounds of the lipophilic phase
the polymer content corresponds to the sum of the amounts of anionic and cationic hydrophilic polymer used
measurement of the particle size using a laser particle size analyzer of Malvern type
assay of the active principle (adapalene or compound A) by HPLC after destruction of the microcapsules using 0.1 N sodium hydroxide solution at 80° C. for 1 hour

### Example 3

### Composition of Microcapsules of Adapalene Encapsulated in the Solid State

In order to obtain adapalene microcapsules, the following ingredients were used in the following proportions:

| | Composition (% w/w) |
|---|---|
| Ingredients | No. 1 |
| Gelatin of type A | 5.35 |
| Gum arabic | 5.35 |
| Adapalene | 0.80 |
| Purified water | qs 100 |

According to the process described in Example 1, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the adapalene microcapsule composition is as follows:

| Characterizations | Results |
|---|---|
| Solids concentration (% w/w capsules) | 11.80 |
| Water content (% w/w Karl Fischer) | 88.50 |
| Particle size (laser particle size analyzer) | D₅₀ = 13.7 μm |
| | D₉₀ = 25.7 μm |

### Example 4

### Composition of Microcapsules of Compound A Encapsulated in the Solid State

In order to obtain compound A microcapsules, the following ingredients were used in the following proportions:

| | Composition (% w/w) |
|---|---|
| Ingredients | No. 2 |
| Gelatin of type A | 3.83 |
| Gum arabic | 3.83 |
| Compound A | 0.04 |
| Purified water | qs 100 |

According to the process described in Example 1, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the compound A microcapsule composition is as follows:

| Characterizations | Results |
|---|---|
| Solids concentration (% w/w capsules) | 7.70 |
| Water content (% w/w Karl Fischer) | 92.30 |
| Particle size (laser particle size analyzer) | D₅₀ = 13.5 μm |
| | D₉₀ = 25.0 μm |

### Example 5

### Composition of Microcapsules of Adapalene Dispersed in the Fatty Phase

In order to obtain adapalene microcapsules dispersed in fatty phase, the following ingredients were used in the following proportions:

| | Composition (% w/w) | |
|---|---|---|
| Ingredients | No. 3 | No. 4 |
| Gelatin of type A | 5.55 | 5.45 |
| Gum arabic | 5.55 | 5.45 |
| Capric/caprylic triglycerides | 40.30 | 29.1 |
| Adapalene | 4.00 | 5.20 |
| Purified water | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the adapalene microcapsule composition is as follows:

| | Results | |
|---|---|---|
| Characterizations | No. 3 | No. 4 |
| Solids concentration (% w/w capsules) | 55.40 | 45.30 |
| Water content (% w/w Karl Fischer) | 44.60 | 54.69 |
| Particle size (laser particle size analyzer) | D₅₀ = 13.1 μm | D₅₀ = 26 μm |
| | D₉₀ = 22.2 μm | D₉₀ = 38 μm |

### Example 6

### Compositions of Microcapsules of Adapalene Dispersed in the Fatty Phase

In order to obtain adapalene microcapsules dispersed in fatty phase, the following ingredients were used in the following proportions:

| | Compositions (% w/w) | | |
|---|---|---|---|
| Ingredients | No. 5 | No. 6 | No. 7 |
| Gelatin of type A | 5.60 | 6.90 | 5.30 |
| Gum arabic | 5.60 | 6.90 | 5.30 |
| Capric/caprylic triglycerides | 29.90 | 30.80 | 35.40 |
| Adapalene | 5.40 | 5.50 | 6.40 |
| Purified water | qs 100 | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the adapalene microcapsule composition is as follows:

| | Results | | |
|---|---|---|---|
| Characterizations | No. 5 | No. 6 | No. 7 |
| Solids concentration | 46.5 | 50.2 | 52.4 |
| (% w/w capsules) | | | |
| Water content (% w/w Karl | 53.5 | 49.8 | 47.6 |
| Fischer) | | | |
| Polymer content (% w/w) | 11.2 | 13.8 | 10.6 |
| Oil content (% w/w) | 29.9 | 30.8 | 35.4 |
| Particle size (laser particle size | D₅₀ = 19 μm | D₅₀ = 31 μm | D₅₀ = 34 μm |
| analyzer) | D₉₀ = 28 μm | D₉₀ = 51 μm | D₉₀ = 75 μm |
| | D₉₉ = 39 μm | D₉₉ = 69 μm | D₉₉ = 95 μm |
| Adapalene concentration | 3.9 | 6.5 | 5.0 |
| (% w/w HPLC) | | | |
| Polymers/oil ratio | 0.37 | 0.45 | 0.30 |

### Example 7

### Composition of Microcapsules of Compound A Dispersed in the Fatty Phase

In order to obtain compound A microcapsules dispersed in fatty phase, the following ingredients were used in the following proportions:

| | Compositions (% w/w) | | |
|---|---|---|---|
| Ingredients | No. 8 | No. 9 | No. 10 |
| Gelatin of type A | 6.1 | 5.1 | 6.9 |
| Gum arabic | 6.1 | 5.1 | 6.9 |
| Liquid paraffin (Primol 352) | 32.3 | 34.2 | 30.7 |
| Compound A | 0.12 | 0.10 | 0.10 |
| Purified water | qs 100 | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the compound A microcapsule composition is as follows:

Results

| Characterizations | No. 8 | No. 9 | No. 10 |
|---|---|---|---|
| Solids concentration | 43.9 | 46.0 | 44.5 |
| (% w/w capsules) | | | |
| Water content | 56.1 | 54.0 | 55.5 |
| (% w/w Karl Fischer) | | | |
| Polymer content (% w/w) | 11.8 | 10.5 | 13.7 |
| Oil content | 31.6 | 35.0 | 30.4 |
| (% w/w) | | | |
| Particle size | D₅₀ = 29 μm | D₅₀ = 31 μm | D₅₀ = 25 μm |
| (laser particle size analyzer) | D₉₀ = 49 μm | D₉₀ = 51 μm | D₉₀ = 37 μm |
| | D₉₉ = 69 μm | D₉₉ = 69 μm | D₉₉ = 51 μm |
| Compound A concentration | 0.60 | 0.66 | 0.51 |
| (% w/w HPLC) | | | |
| Polymers/oil ratio | 0.37 | 0.30 | 0.45 |

### Example 8

### Compositions of Microcapsules of Compound A Dissolved in the Fatty Phase

In order to obtain compound A microcapsules dissolved in fatty phase, the following ingredients were used in the following proportions:

| | Compositions (% w/w) | | |
|---|---|---|---|
| Ingredients | No. 11 | No. 12 | No. 13 |
| Gelatin of type A | 6.1 | 5.2 | 7.0 |
| Gum arabic | 6.1 | 5.2 | 7.0 |
| Capric/caprylic triglycerides | 24.0 | 25.5 | 22.9 |
| Phenoxyethanol | 8.6 | 8.9 | 8.0 |
| Compound A | 0.15 | 0.16 | 0.15 |
| Purified water | qs 100 | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

After drying, the compound A microcapsule composition is as follows:

| | Results | | |
|---|---|---|---|
| Characterization | No. 11 | No. 12 | No. 13 |
| Solids concentration (% w/w capsules) | 44.0 | 45.0 | 44.0 |
| Water content (% w/w Karl Fischer) | 56.0 | 55.0 | 56.0 |
| Polymer content (% w/w) | 12.2 | 10.4 | 13.6 |
| Oil content (% w/w) | 32.6 | 34.4 | 30.9 |
| Particle size (laser particle size analyzer) | D50 = 5 μm | D50 = 13 μm | D50 = 6 μm |
| | D90 = 9 μm | D90 = 25 μm | D0 = 13 μm |
| | D99 = 15 μm | D99 = 43 μm | D99 = 21 μm |
| Compound A concentration (% w/w HPLC) | 0.12 | 0.17 | 0.14 |
| Polymers/oil ratio | 0.37 | 0.30 | 0.44 |

### Example 9

### Compositions of Microcapsules of Compound A Dissolved in the Fatty Phase

In order to obtain compound A microcapsules dissolved in fatty phase, the following ingredients were used in the following proportions:

| | Compositions (% w/w) | |
|---|---|---|
| Ingredients | No. 14 | No. 15 |
| Gelatin of type A | 6.0 | 5.3 |
| Gum arabic | 6.0 | 5.3 |
| PPG-15 stearyl ether | 32.5 | 34.4 |
| Compound A | 0.20 | 0.21 |
| Purified water | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

At the end of preparation, 0.5% of phenoxyethanol was added to the preparation.

After drying, the compound A microcapsule composition is as follows:

| | Results | |
|---|---|---|
| Characterization | No. 14 | No. 15 |
| Solids concentration (% w/w capsules) | 44.0 | 46.0 |
| Water content (% w/w Karl Fischer) | 56.0 | 54.0 |
| Polymer content (% w/w) | 11.9 | 10.6 |
| Oil content (% w/w) | 31.8 | 35.2 |
| Particle size (laser particle size analyzer) | D₅₀ = 13 μm | D₅₀ = 15 μm |
| | D₉₀ = 22 μm | D₉₀ = 25 μm |
| | D₉₉ = 39 μm | D₉₉ = 39 μm |
| Compound A concentration (% w/w HPLC) | 0.20 | 0.24 |
| Polymers/oil ratio | 0.37 | 0.30 |

### Example 10

### Compositions of Microcapsules of Compound A Dissolved in the Fatty Phase

In order to obtain compound A microcapsules dissolved in fatty phase, the following ingredients were used in the following proportions:

| | Compositions (% w/w) | | |
|---|---|---|---|
| Ingredients | No. 16 | No. 17 | No. 18 |
| Gelatin of type A | 6.1 | 5.2 | 6.9 |
| Gum arabic | 6.1 | 5.2 | 6.9 |
| PPG-15 stearyl ether | 25.2 | 27.5 | 23.1 |
| Phenoxyethanol | 7.4 | 7.0 | 7.8 |
| Compound A | 0.20 | 0.20 | 0.20 |
| Purified water | qs 100 | qs 100 | qs 100 |

According to the process described in Example 2, the pH was adjusted to 4.9 with acetic acid. In order to enable crosslinking, an amount of glutaraldehyde corresponding to 16% of the total amount of polymers used was added.

After drying, the compound A microcapsule composition is as follows:

| | Results | | |
|---|---|---|---|
| Characterization | No. 16 | No. 17 | No. 18 |
| Solids concentration (% w/w capsules) | 45.0 | 45.5 | 41.0 |
| Water content (% w/w Karl Fischer) | 55.0 | 54.5 | 59.0 |
| Polymer content (% w/w) | 12.2 | 10.44 | 12.65 |
| Oil content (% w/w) | 32.54 | 34.80 | 28.12 |
| Particle size (laser particle size analyzer) | D₅₀ = 7 μm | D₅₀ = 13 μm | D₅₀ = 5 μm |
| | D₉₀ = 14 μm | D₉₀ = 27 μm | D₉₀ = 11 μm |
| | D₉₉ = 21 μm | D₉₉ = 43 μm | D₉₉ = 24 μm |
| Compound A concentration (% w/w HPLC) | 0.18 | 0.24 | 0.21 |
| Polymers/oil ratio | 0.37 | 0.30 | 0.45 |

### Example 11

### Composition and Stability of a Gel Containing Microcapsules of Adapalene Dispersed in a Fatty Phase

The composition of the type comprising No. 5 microcapsules was prepared and its stability monitored for three months and under three temperature conditions: +4° C., room temperature and 40° C. At each checkpoint, the following characterizations were performed:
The macroscopic observation is performed on the formulation in its original packaging.
The microscopic observation is performed using an Axio.Scope A1 microscope (polarized light, objective ×20).
The pH measurement is taken in the formulation.
The viscosity measurement is performed using a machine such as a Brookfield RVDVII+ viscometer.

Depending on the physical appearance of the composition, the operating conditions such as the choice of needle and of speed may vary. The measurements are performed after 1 minute, in the original packaging (250 ml wide-aperture jars).

In order to monitor the chemical stability of the compositions, the adapalene titer is checked by HPLC after preparation (T0) and after 1 month, 2 months and 3 months, at two storage temperatures: room temperature and 40° C.:
The results at T0 are expressed in mg/g.
The results at each analytical checkpoint (T1M, T2M, T3M) are expressed as %/T0.

| Ingredients | Composition (% w/w) |
|---|---|
| Disodium edetate | 0.10 |
| Glycerol | 4.0 |
| Propylene glycol | 4.0 |
| Sodium docusate | 0.05 |
| Microcapsules No. 5 | 7.69 |
| Poloxamer 124 | 0.20 |
| Acrylamide/AMPS | 4.0 |
| copolymer | |
| dispersion 40%/ | |
| isohexadecane | |
| Purified water | qs 100 |

Characterizations at the initial time
Smooth glossy light white gel
pH: 4.44
Brookfield viscosity (No. 27 needle, speed 2.5 rpm): 88 000 cP

| | | RT | 4° C. | 40° C. |
|---|---|---|---|---|
| Macroscopic | T1M | Complies | Complies | Slightly ivory- |
| appearance | | | | colored |
| | T2M | Complies | Complies | Slightly ivory- |
| | | | | colored |
| | T3M | Complies | Complies | Slightly ivory- |
| | | | | colored |
| Brookfield | T1M | 91600 | NA | 85200 |
| viscosity | T2M | 95200 | NA | 91400 |
| | T3M | 95300 | NA | 83400 |
| pH | T1M | 4.01 | 4.27 | 3.41 |
| | T2M | 3.78 | 4.25 | 3.38 |
| | T3M | 3.70 | 4.30 | 3.46 |
| Dosage (mg/g) | T0 | 2.838 | — | — |
| Adapalene | T1M | ND | ND | 106.3 |
| % T0 | T2M | 102.2 | ND | ND |
| | T3M | 104.4 | ND | 102.6 |

ND: not done

The stability results show that the gel comprising No. 5 adapalene microcapsules is physically and chemically stable.

### Example 12

### Composition and Stability of a Gel Containing Microcapsules of Compound A Dispersed in a Fatty Phase

The composition of the type comprising No. 8 microcapsules was prepared and its stability monitored for three months and under three temperature conditions: +4° C., room temperature and 40° C. At each checkpoint, the following characterizations were performed:
The macroscopic observation is performed on the formulation in its original packaging.
The microscopic observation is performed using an Axio.Scope A1 microscope (polarized light, objective ×20).
The pH measurement is taken in the formulation.
The viscosity measurement is performed using a machine such as a Brookfield RVDVII+ viscometer.

Depending on the physical appearance of the composition, the operating conditions such as the choice of needle and of speed may vary. The measurements are performed after 1 minute, in the original packaging (250 ml wide-aperture jars).

In order to monitor the chemical stability of the compositions, the compound A titer is checked by HPLC after preparation (T0) and after 1 month, 2 months and 3 months, at two storage temperatures: room temperature and 40° C.:
The results at T0 are expressed in mg/g.
The results at each analytical checkpoint (T1M, T2M, T3M) are expressed as %/T0.

| Ingredients | Composition (% w/w) |
|---|---|
| Sodium docusate | 0.05 |
| Sodium edetate | 0.10 |
| Methyl paraben | 0.20 |
| Glycerol | 4.00 |
| 1,2-Propanediol | 4.00 |
| Poloxamer 124 | 0.20 |
| No. 8 compound A | 1.66 |
| microcapsules | |
| Acrylamide/AMPS | 4.00 |
| copolymer | |
| dispersion 40%/ | |
| isohexadecane | |
| Purified water | qs 100 |

Characterization at the initial time
Glossy white gel
Microscopic observation: Many small microcapsules 2 to 20 μm
Brookfield viscosity (No. 29 needle, speed 5 rpm): 104 000 cP
pH: 4.66

| | | RT | 4° C. | 40° C. |
|---|---|---|---|---|
| Macroscopic | T1M | Complies | Complies | Complies |
| appearance | T2M | ND | ND | ND |
| | T3M | Complies | Complies | Complies |
| Microscopic | T1M | Complies | Complies | Complies |
| appearance | T2M | ND | ND | ND |
| | T3M | Complies | Complies | Complies |
| Brookfield | T1M | 104 000 | NA | 102 000 |
| viscosity | T2M | ND | NA | ND |
| | T3M | 106 000 | NA | 104 000 |
| pH | T1M | 470 | 471 | 472 |
| | T2M | ND | ND | ND |
| | T3M | 4.66 | 4.76 | 4.63 |
| Dosage (mg/g) | T0 | 0.00954 | — | — |
| Compound A | T1M | 99.60 | ND | 100.70 |
| %/T0 | T2M | 101.47 | ND | 99.60 |
| | T3M | 105.70 | ND | 103.10 |

ND: not done

The stability results show that the gel comprising No. 8 compound A microcapsules is physically and chemically stable.

### Example 13

### Composition and Stability of a Gel Containing Microcapsules of Compound A Dissolved in a Fatty Phase

The composition of the type comprising No. 14 microcapsules was prepared and its stability monitored for three months and under three temperature conditions: +4° C., room temperature and 40° C. At each checkpoint, the following characterizations were performed:
The macroscopic observation is performed on the formulation in its original packaging.
The microscopic observation is performed using an Axio.Scope A1 microscope (polarized light, objective ×20).
The pH measurement is taken in the formulation.
The viscosity measurement is performed using a machine such as a Brookfield RVDVII+ viscometer.

Depending on the physical appearance of the composition, the operating conditions such as the choice of needle and of speed may vary. The measurements are performed after 1 minute, in the original packaging (250 ml wide-aperture jars).

In order to monitor the chemical stability of the compositions, the compound A titer is checked by HPLC after preparation (T0) and after 1 month, 2 months and 3 months, at two storage temperatures: room temperature and 40° C.:
The results at T0 are expressed in mg/g.
The results at each analytical checkpoint (T1M, T2M, T3M) are expressed as %/T0.

| Ingredients | Composition (% w/w) |
|---|---|
| Sodium docusate | 0.05 |
| Sodium edetate | 0.10 |
| Methyl paraben | 0.20 |
| Glycerol | 4.00 |
| 1,2-Propanediol | 4.00 |
| Poloxamer 124 | 0.20 |
| No. 14 compound A | 4.16 |
| microcapsules | |
| Acrylamide/ | 4.00 |
| AMPS copolymer | |
| dispersion 40%/ | |
| isohexadecane | |
| Purified water | qs 100 |

Characterization at the initial time
Glossy white gel
Microscopic observation: Many microcapsules 10 to 20 μm
Brookfield viscosity (No. 29 needle, speed 5 rpm): 127 000 cP
pH: 4.98

| | | RT | 4° C. | 40° C. |
|---|---|---|---|---|
| Macroscopic | T1M | Complies | Complies | Complies |
| appearance | T2M | ND | ND | ND |
| | T3M | Complies | Complies | Complies |
| Microscopic | T1M | Complies | Complies | Complies |
| appearance | T2M | ND | ND | ND |
| | T3M | Complies | Complies | Complies |
| Brookfield | T1M | 127 000 | ND | 124 000 |
| viscosity | T2M | ND | ND | ND |
| | T3M | 126 000 | ND | 125 000 |
| pH | T1M | 4.59 | 4.68 | 4.62 |
| | T2M | ND | ND | ND |
| | T3M | 4.63 | 4.68 | 4.55 |
| Dosage (mg/g) | T0 | 0.01044 | — | — |
| Compound A | T1M | 96.50 | ND | 95.70 |
| %/T0 | T2M | 95.70 | ND | 95.70 |
| | T3M | 98.70 | ND | 99.80 |

ND: not done

The stability results show that the gel comprising No. 14 compound A microcapsules is physically and chemically stable.

### Example 14

### Composition and Stability of a Cream Containing Microcapsules of Compound A Dissolved in a Fatty Phase

The composition of the type comprising No. 16 microcapsules was prepared and its stability monitored for three months and under three temperature conditions: +4° C., room temperature and 40° C. At each checkpoint, the following characterizations were performed:
The macroscopic observation is performed on the formulation in its original packaging.
The microscopic observation is performed using an Axio.Scope A1 microscope (polarized light, objective ×20).
The pH measurement is taken in the formulation.
The viscosity measurement is performed using a machine such as a Brookfield RVDVII+ viscometer.

Depending on the physical appearance of the composition, the operating conditions such as the choice of needle and of speed may vary. The measurements are performed after 1 minute, in the original packaging (250 ml wide-aperture jars).

In order to monitor the chemical stability of the compositions, the compound A titer is checked by HPLC after preparation (T0) and after 1 month, 2 months and 3 months, at two storage temperatures: room temperature and 40° C.:
The results at T0 are expressed in mg/g.
The results at each analytical checkpoint (T1M, T2M, T3M) are expressed as %/T0.

| Ingredients | Composition (% w/w) |
|---|---|
| Allantoin | 0.2 |
| Sodium docusate | 0.05 |
| Sodium edetate | 0.10 |
| Methyl paraben | 0.20 |
| Glycerol | 2.00 |
| 1,2-Propanediol | 3.00 |
| Poloxamer 124 | 0.10 |
| Talc PH | 2.00 |
| Xanthan gum | 0.50 |
| Lactic acid solution | 8.00 |
| (1% w/w) | |
| No. 16 compound A | 5.55 |
| microcapsules | |
| Cyclomethicone 5 | 8.00 |
| Dimethicone 350 cSt | 1.00 |
| Liquid paraffin oil | 1.00 |
| Phenoxyethanol | 0.80 |
| Acrylamide/ | 4.00 |
| AMPS copolymer | |
| dispersion 40%/ | |
| isohexadecane | |
| Purified water | qs 100 |

Characterization at the initial time
Glossy white cream
Microscopic observation: many microcapsules from 5 μm to 20 μm
Brookfield viscosity (No. 6 needle, speed 2 rpm): 185 000 cP
pH: 4.98

| | | RT | 4° C. | 40° C. |
|---|---|---|---|---|
| Macroscopic | T1M | Complies | Complies | Complies |
| appearance | T2M | ND | ND | ND |
| | T3M | Complies | Complies | Slightly ivory- |
| | | | | colored |
| Microscopic | T1M | Complies | Complies | Complies |
| appearance | T2M | ND | ND | ND |
| | T3M | Complies | Complies | Complies |
| Brookfield | T1M | 166 000 | ND | 158 000 |
| viscosity | T2M | ND | ND | ND |
| | T3M | 166 000 | ND | ND |
| pH | T1M | 5.10 | 5.12 | 5.03 |
| | T2M | ND | ND | ND |
| | T3M | 5.06 | 5.06 | 4.94 |
| Dosage (mg/g) | T0 | 0.01052 | — | — |
| Compound A | T1M | 101.00 | ND | 95.44 |
| %/T0 | T2M | 99.90 | ND | 102.90 |
| | T3M | ND | ND | 101.6 |

ND: not done

The stability results show that the cream comprising No. 16 compound A microcapsules is physically and chemically stable.

### Example 15

### Profile of In Vitro Release of Compound A From Microcapsules

The release kinetics of compound A from microcapsules was evaluated on 24-well microplates (Corning HTS Transwell plate) having a polyester membrane. About 200 mg of test composition were deposited on this membrane. The receiving phase is composed of a propylene glycol/ethanol mixture (20/80) allowing good dissolution of compound A.

Each plate was shaken during the analysis and samples were taken regularly at 0.5 h; 1 h; 2 h; 3 h; 4 h; 5 h and 24 h. The assay of compound A was performed by HPLC.

The release kinetics of compound A from the composition of Example 13 was studied in comparison with a glycol-alcohol reference gel comprising dissolved but non-encapsulated compound A. For each composition, the release kinetics were studied in triplicate.

The values obtained using the reference gel are the following:

| | Square | | | | |
|---|---|---|---|---|---|
| | root of | Amount | Coefficient | Amount | Coefficient |
| Time | the time | released | of variation | released | of variation |
| (h) | (h^{1/2}) | (μg/cm²) | (μg/cm²) | (%) | (%) |
| 0.5 | 0.71 | 1.9296 | 0.0541 | 3.64 | 0.1052 |
| 1 | 1.00 | 3.9003 | 0.1676 | 7.36 | 0.2759 |
| 2 | 1.41 | 6.2557 | 0.2887 | 11.80 | 0.4400 |
| 3 | 1.73 | 8.9024 | 0.3043 | 16.80 | 0.8052 |
| 4 | 2.00 | 11.3779 | 0.3502 | 21.46 | 0.3297 |
| 5 | 2.24 | 13.7134 | 0.6812 | 25.86 | 0.6447 |
| 24 | 4.90 | 44.2732 | 1.9839 | 83.49 | 2.1664 |

The values obtained for Example 13 are the following:

| | Square | | | | |
|---|---|---|---|---|---|
| | root of | Amount | Coefficient | Amount | Coefficient |
| Time | the time | released | of variation | released | of variation |
| (h) | (h^{1/2}) | (μg/cm²) | (μg/cm²) | (%) | (%) |
| 0.5 | 0.71 | 0.2837 | 0.2159 | 0.45 | 0.3265 |
| 1 | 1.00 | 0.7622 | 0.5340 | 1.21 | 0.8041 |
| 2 | 1.41 | 1.7692 | 0.7359 | 2.82 | 1.0716 |
| 3 | 1.73 | 3.4933 | 0.6958 | 5.59 | 0.9207 |
| 4 | 2.00 | 5.4971 | 0.5041 | 8.83 | 0.5568 |
| 5 | 2.24 | 8.1934 | 0.3317 | 13.17 | 0.4253 |
| 24 | 4.90 | 41.8027 | 5.2623 | 67.40 | 10.4155 |

FIG. 1 shows the amount released in percentage of compound A as a function of the square root of the time from the reference gel and from the composition of Example 13.

Comparison of the curves shows that the release profile of compound A from the microcapsules is different from that for the dissolved non-encapsulated compound A. Specifically, the release profile of the microcapsules is nonlinear and shows two release phases:
a slow release during the first stages of the study between 0 and 2 hours, and
a faster release from 2 hours up to 24 hours, the end of the study.

FIGS. 2 and 3 show, respectively, the amount of compound A expressed in μg/cm² as a function of the square root of the time. From the curves obtained, the linear regressions were determined between 0 and 2 hours and between 2 and 24 hours so as to calculate the release constants for each time interval.

The various parameters calculated are given in the table below:
the release constant calculated from the linear regression determined for each release profile
the lag time calculated from the linear regression corresponding to the time interval 0-2 h
the point of inflection corresponding to the intersection of the two linear regressions

| | Release | Release | Point of | |
|---|---|---|---|---|
| Compo- | constant 0-2 h | constant 2-24 h | inflection | Lag time |
| sitions | (μg/cm²/h^{1/2}) | (μg/cm²/h^{1/2}) | (hours) | (hours) |
| Reference | 6.085 | 11.102 | 2.58 | 0.14 |
| Example 13 | 2.1228 | 11.926 | 2.62 | 0.36 |

Comparison of the results shows that the microcapsules of compound A have release kinetics with a delay effect during the first two hours of the release study. Specifically, the lag time before release is about 2.5 times longer than that for the dissolved non-encapsulated compound A.

Furthermore, in this same time interval, the release kinetics of compound A from the microcapsules is about 3 times slower compared with that of the dissolved non-encapsulated compound A.

On the other hand, from 2 hours, the release profile of compound A is identical whether or not it is encapsulated. Specifically, the release constants between 2 and 24 hours are very similar.

The encapsulation of compound A using the system as proposed by the invention offers the advantage of reducing any risk of irritation caused by compound A during the first hours after application, since the amount of retinoid released is smaller. As a result, less absorption takes place and the risks of irritation are reduced.

On the other hand, after the first hours following the application, the fact that the same release kinetics are obtained at the long times shows that compound A is available to the absorbed by the skin tissues.

The irritation caused by compound A may thus be modulated without, however, having an impact on the profile of absorption of compound A after 2 hours of application.

The microcapsules as defined by the invention also have the advantage of having a short-lasting delay effect on the release kinetics of compound A.

### Example 16

### Tolerance Study: Evaluation of the Pro-Inflammatory Effect of the Formulations After Repeated Application to the Ear of BALBB/C Mice

The aim of this study is to study the irritant effect of compound A encapsulated in microcapsules obtained by coacervation according to the invention.

A repeated application of 3 mg of each test composition was administered to the ear of the mice on day 1 and for 4 weeks. Clinical observations and measurements of the mouse ear thickness directly linked to inflammation are performed from day 2 and daily up to day 26.

The results are expressed by calculating the area under the curve obtained from the graph representing the change in ear thickness in the course of the study.

A Student statistical test was performed for each test composition versus the reference gel so as to demonstrate the significant differences between the various results obtained.

A placebo composition of gel type was prepared and using which an amount of microcapsules of compound A was introduced so as to obtain a compound A content of 0.01% by weight relative to the the weight of the final composition. The microcapsules tested correspond to those described in Examples 8, 9, 13, 14, 16 and 18.

The composition of placebo gel type is as follows:

| Ingredients | Composition (% w/w) |
|---|---|
| Sodium docusate | 0.05 |
| Sodium edetate | 0.10 |
| Methyl paraben | 0.20 |
| Glycerol | 4.00 |
| 1,2-Propanediol | 4.00 |
| Poloxamer 124 | 0.20 |
| Acrylamide/ | 4.00 |
| AMPS copolymer | |
| dispersion 40%/ | |
| isohexadecane | |
| Purified water | qs 100 |

A glycol-alcohol reference gel in which the compound A is dissolved but not encapsulated was used.

FIG. 4 represents the various area under the curve values obtained for each test composition.

It emerges from these results that compound A dispersed or dissolved in the microcapsules is less irritant than compound A dissolved in a reference gel.

The decrease in irritation due to compound A versus the reference is greater with the microcapsules containing dispersed compound A (No. 8 and No. 9).

The decrease in irritation due to compound A versus the reference obtained with microcapsules No. 13, 14, 16 and 18 in which compound A is dissolved is less pronounced.

The microcapsules of compound A obtained by coacervation make it possible to reduce the irritation to a greater or lesser extent according to the presentation form of compound A: dispersed or dissolved within these microcapsules.

1. A microcapsule, comprising:
a pharmaceutical active agent chosen from retinoids;
a cationic hydrophilic polymer chosen from gelatins of type A; and
an anionic hydrophilic polymer.

2. The microcapsule as claimed in claim 1, wherein the microcapsule also comprises a lipophilic phase.

3. The microcapsule as claimed in claim 1, wherein the retinoid is selected from the group consisting of all-trans-retinoic acid or tretinoin, 13-cis-retinoic acid or isotretinoin, acitretin, arotinoic acid, retinol, adapalene, tazarotene, retinaldehyde, etretinate, 3″-tert-butyl-4′-(2-hydroxyethoxy)-4″-pyrrolidin-1-yl-[1,1′;3′,1″]-terphenyl-4-carboxylic acid, 2-hydroxy-4-[3-hydroxy-3-(5,6,7,8-tetrahydro-5,5,8,8-tetramethyl-2-naphthyl)-1-propynyl]benzoic acid or an enantiomer thereof, 4′-(4-isopropylamino-butoxy)-3′-(5,5,8,8-tetramethyl-5,6,7,8-tetrahydronaphthalen-2-yl)-biphenyl-4-carboxylic acid, 4-{3-hydroxy-3-[4-(2-ethoxyethoxy)-5,5,8,8-tetramethyl-5,6,7,8-tetrahydronaphthalen-2-yl]-prop-1-ynyl}benzoic acid and 4-[2-(3-tert-butyl-4-diethylaminophenyl)-2-hydroxyiminoethoxy]-2-hydroxybenzoic acid.

4. The microcapsule as claimed in claim 3, wherein the retinoid is 3″-tert-butyl-4′-(2-hydroxyethoxy)-4″-pyrrolidin-1-yl-[1,1′;3′,1″]-terphenyl-4-carboxylic acid or adapalene.

5. The microcapsule as claimed in claim 1, wherein the anionic hydrophilic polymer is gum arabic.

6. The microcapsule as claimed in claim 1, wherein the pharmaceutical active agent is encapsulated directly in the solid state, or dispersed in a lipophilic phase, or dissolved in a lipophilic phase.

7. The microcapsule as claimed in claim 1, it wherein the microcapsule comprises:
3″-tert-butyl-4′-(2-hydroxyethoxy)-4″-pyrrolidin-1-yl[1,1′;3′,1″]terphenyl-4-carboxylic acid,
gelatin of type A, and
gum arabic.

8. A microcapsule comprising:
3″-tert-butyl-4′-(2-hydroxyethoxy)-4″-pyrrolidin-1-yl-[1,1′;3′,1″]-terphenyl-4-carboxylic acid as active agent, in solid form or in dispersed form in a lipophilic phase;
a cationic hydrophilic polymer, and
an anionic hydrophilic polymer,
and wherein the area under a curve, determined by applying to the ears of mice, once a day for 4 consecutive weeks, 3 mg of a composition comprising said microcapsules, such that the content of 3″-tert-butyl-4′-(2-hydroxyethoxy)-4″-pyrrolidin-1-yl-[1,1′;3′,1″]-terphenyl-4-carboxylic acid is 0.01% by weight relative to the total weight of the composition, and by measuring the thickness of the mouse ear from day 2 and then daily up to day 26, and plotting the corresponding graph representing the change in thickness of the ear over time and calculating the area under this curve, is less than 2000 μm per day.

9. A microcapsule comprising:
3″-tert-butyl-4′-(2-hydroxyethoxy)-4″-pyrrolidin-1-yl-[1,1′;3′,1′]-terphenyl-4-carboxylic acid as active agent, in dissolved form in a lipophilic phase;
a cationic hydrophilic polymer; and
an anionic hydrophilic polymer,
and wherein the area under a curve, determined by applying to the ears of mice, once a day for 4 consecutive weeks, 3 mg of a composition comprising said microcapsules, such that the content of 3″-tert-butyl-4′-(2-hydroxyethoxy)-4″-pyrrolidin-1-yl-[1,1′;3′,1″]-terphenyl-4-carboxylic acid is 0.01% by weight relative to the total weight of the composition, and by measuring the thickness of the mouse ear from day 2 and then daily up to day 26, and plotting the corresponding graph representing the change in thickness of the ear over time and calculating the area under this curve, is less than 4000 μm per day.

10. A topical pharmaceutical composition comprising the microcapsule as claimed in claim 1.

11. The composition as claimed in claim 10, wherein the composition is in the form of an emulsion, a suspension or a gel.

12. A process for preparing the microcapsule defined in claim 1, the process comprising the following steps:
dissolving two oppositely charged hydrophilic polymers;
adding the retinoid and mixing;
adding a pH regulator to a coacervation pH;
adding an agent for crosslinking the two polymers;
drying resulting microcapsules;
removing the crosslinking agent by washing with a saline solution; and
successive washing of the preparation with water and drying.

13. A process for preparing the microcapsule as claimed in claim 1, in which the retinoid is in the solid state, said process comprising the following steps:
heating dilution water to 40° C. in a reactor
preparing a gum arabic solution in a formulation beaker of suitable size; and
dispersing the retinoid in this phase and heating to 40° C.;
preparing an aqueous solution of gelatin of type A in a second beaker and heating 40° C.;
with stirring, gently pouring the solution of gelatin of type A into the aqueous solution of gum arabic comprising the dispersed retinoid;
stirring until the mixture is fully homogeneous;
performing dilution in the reactor, with the dilution water at 40° C.;
with stirring, adding acetic acid to the preparation in an amount sufficient to descend to a coacervation pH;
reducing the temperature to 10° C.;
solidifying coacervates by adding the crosslinking agent;
drying at 50° C.;
recovering and washing resulting capsules in a specific saline solution;
washing twice more with water so as to remove residual salts;
adding preserving agent to the preparation; and
drying the coacervates under a gentle vacuum to obtain a manipulable capsule paste.

14. A process for preparing the microcapsule as claimed in claim 1, in which the retinoid is dispersed or dissolved, said process comprising the following steps:
heating dilution water to 40° C. in a reactor;
preparing an aqueous solution of polymer in a formulation beaker of suitable size and heating the mixture to 40° C.;
in a second beaker, dispersing or dissolving the retinoid in the fatty phase and hearing to 40° C.;
with stirring, gently pouring the oily phase comprising the retinoid into the aqueous solution of polymer and stirring until the mixture is fully homogeneous (emulsification);
diluting the emulsion in the reactor, with the dilution water at 40° C.;
with stirring, adding acetic acid to the emulsion in an amount sufficient to descend to a coacervation;
reducing the temperature to 10° C.;
solidifying coacervates by adding crosslinking agent;
drying at 50° C.;
recovering and washing resulting microcapsules in a specific saline solution so as to remove residual crosslinking agent;
washing twice more with water to remove residual salts;
adding preserving agent to the preparation; and
drying coacervates under a gentle vacuum to obtain a manipulable microcapsule paste.

15. A method of treating a pathology, the method comprising administering to an individual subject in need thereof an effective amount of composition as claimed in claim 10, wherein the pathology being treated is one or more pathologies selected from the group consisting of:
1) dermatological conditions associated with a keratinization disorder relating to cell differentiation and proliferation, in particular for treating common acne, comedonal acne, polymorphic acne, acne rosacea, nodulocystic acne, acne conglobata, senile acne, secondary acne such as solar acne, acne medicamentosa or occupational acne;
2) keratinization disorders, in particular ichthyosis, ichthyosiform conditions, lamellar ichthyosis, Darier's disease, palmoplantar keratoderma, leukoplakia, pityriasis rubra pilaris and leukoplakiform conditions, cutaneous or mucosal (buccal) lichen;
3) dermatological conditions with an inflammatory immuno-allergic component, with or without a cell proliferation disorder, and in particular all forms of psoriasis, whether cutaneous, mucosal or ungual, and even psoriatic arthritis, or else atopic dermatitis and the various forms of eczema;
4) skin disorders caused by exposure to UV radiation, and also for repairing or combating skin aging, whether it is photo-induced or chronological, or for reducing actinic keratoses and pigmentations, or any pathological conditions associated with chronological or actinic aging, such as xerosis, pigmentations and wrinkles;
5) conditions associated with benign dermal or epidermal proliferations, whether or not they are of viral origin, such as common warts, flat warts, molluscum contagiosum and epidermodysplasia verruciformis, or oral or florid papillomatoses;
6) dermatological disorders such as immune dermatoses, for instance lupus erythematosus, bullous immune diseases and collagen diseases, such as scleroderma;
7) stigmata of epidermal and/or dermal atrophy induced by local or systemic corticosteroids, or any other form of cutaneous atrophy;
8) cicatrization disorders, or for preventing or repairing stretch marks, or else for promoting cicatrization;
9) skin disorders of fungal origin, such as tinea pedis and tinea versicolor;
10) pigmentation disorders, such as hyperpigmentation, melasma, hypopigmentation or vitiligo; and
11) cutaneous or mucosal cancerous or precancerous conditions, such as actinic keratoses, Bowen's disease, in-situ carcinomas, keratoacanthomas and skin cancers such as basal cell carcinoma (BCC), squamous cell carcinoma (SCC) and cutaneous lymphomas such as T lymphoma.

16. A method of treating acne, the method comprising administering to an individual subject in need thereof an effective amount of the composition comprising the microcapsule as claimed in claim 1.

17. The method as claimed in claim 15, wherein the pathology is ichthyosis, ichthyosiform conditions, palmoplantar keratosis or psoriasis.

18. The microcapsule as claimed in claim 4, wherein the retinoid is 3″-tert-butyl-4′-(2-hydroxyethoxy)-4″-pyrrolidin-1-yl-[1,1′,3′,1″]-terphenyl-4-carboxylic acid
